(19) United States
(12) Patent Application Publication
(10) Pub. No.: US 2026/0345355 A1
(43) Pub. Date: Nov. 5, 2026

(54) METHOD FOR DETERMINING PARAMETERS FOR FITTING A PROSTHESIS ON A JOINT OF A PATIENT

(71) Applicant: TWINSIGHT, Grenoble (FR)

(72) Inventors: Marek Janusz BUCKI, Theys (FR); Mathieu Raymond Albert BAILET, Grenoble (FR); Antoine Pierre-André PERRIER, Yvry-sur-Seine (FR); Mathieu Pierre Yves RIMAUD, Grenoble (FR)

(73) Assignee: TWINSIGHT, Grenoble (FR)

(21) Appl. No.: 19/486,422

(22) PCT Filed: May 24, 2024
(86) PCT No.: PCT/FR2024/050666

§ 371 Date: Nov. 21, 2025

(30) Foreign Application Priority Data
May 24, 2023 (FR) FR2305146

(51) Int. Cl. A61F 2/76; A61F 2/50; G06F 30/27; G06T 7/50; G16H 10/60

(52) U.S. Cl. CPC A61F 2/76; G06F 30/27; G06T 7/50; G16H 10/60; A61F 2002/505; A61F 2002/762; G06T 2200/04; G06T 2207/30008; G06T 2207/30052

(57) ABSTRACT

A method for determining parameters for fitting a prosthesis on joint of patient, method being wherein it comprises implementation, by data-processing means of server, of steps of: (a) obtaining kinematic model of joint before prosthesis is fitted, referred to as preoperative kinematic model; (b) constructing a kinematic model of joint after prosthesis has been fitted, referred to as postoperative kinematic model, for current values of fitting parameters of prosthesis, by modifying preoperative kinematic model; (c) predicting postoperative values of a set of functional metrics of joint depending on postoperative kinematic model; (d) validating or not current values of fitting parameters of prosthesis depending on predicted postoperative values of the set of functional metrics; (e) if current values of fitting parameters of prosthesis are not validated, modifying current values of fitting parameters of prosthesis, updating postoperative kinematic model, then repeating steps (c) and (d) on basis of updated postoperative kinematic model.

### GENERAL TECHNICAL FIELD

The present invention relates to the field of biomechanics. More specifically, it concerns a method for determining parameters for fitting a prosthesis on a joint of a patient.

### STATE OF THE ART

A joint prosthesis is an internal implant (endoprosthesis) that replaces the damaged surfaces of a joint, with the aim of enabling, among other things, stable support, flexion and extension, and pain relief.

The most common example is the knee prosthesis, but there are also numerous hip, shoulder, and other prostheses.

The knee prosthetic surgery has thus undergone rapid evolution in practices, moving from essentially bone surgery to surgery capable of taking into account the surrounding soft tissues in order to guarantee prosthetic longevity and a good quality of life for the patient. Nevertheless, more than 20% of patients report dissatisfaction, mainly due to persistent pain and the inability to resume a number of daily activities.

This high rate of patient dissatisfaction is largely explained by the shortcomings of certain surgical protocols, for example, in taking into account the balancing of tensions in the peripheral ligamentous structures of the knee, and in particular the external and internal collateral ligaments, or in taking into account postoperative patellar kinematics during flexion and extension.

Thus, the document K. A. Gustke, G. J. Golladay, M. W. Roche, G. J. Jerry, L. C. Elson, C. R. Anderson, “Increased satisfaction after total knee replacement using sensor-guided technology”, Bone Joint J 2014; 96-B:1333-8, October 2014 shows that the patient dissatisfaction rate decreases from 21.1% to 3.3% when the surgeon takes ligament balance into account in their protocol.

The problem is that taking into account soft tissues such as ligamentous structures, or for example in the case of the knee, the patellar tracking, during joint surgeries involves very complex personalized biomechanical considerations to guarantee the precision of the surgical procedure.

To take the example of ligament balancing, many approaches have been explored to more easily integrate these aspects into informed therapeutic decision-making:
Some manufacturers focus on the design of polyethylene inserts (particularly for the prosthetic tibial plateau) in order to maximize the congruence between the implants and thus stabilize the joint. This approach partially and only indirectly compensates for the shortcomings of ligament balancing.
So-called «bi-uni» operative strategies (sometimes robotic-assisted) allow fitting unicompartmental prostheses on both sides of the joint (which normally cover only one side—medial or lateral—of the knee) in order to preserve the soft tissues and therefore the stability of the joint. Unfortunately, this is not always possible.
The intraoperative robotics allows assisting the surgeon in measuring intra-articular spaces in extension and flexion in order to balance the bilateral tension of the collateral ligaments. However, this measurement can only be made after the first bone cut (generally tibial cut) has been made, which limits its benefits.

The existing techniques are therefore insufficient, and it remains desirable to obtain a surgical planning method that:
1) accurately takes into account the patient's morphology,
2) allows for accurate simulation of the patient's joint kinematics,
3) proposes parameters for fitting the prosthesis guiding the surgeon towards the best option for the patient, and
4) can be used by the surgeon as early as possible during the surgical procedure to avoid actions such as bone cuts for which there is little or no possibility of correction in case of a change in planning.

Documents WO 2020163324, US2022087822, AU 2021356545, U.S. Pat. No. 8,521,492 and US2005119661 propose methods for determining parameters for fitting a prosthesis by constructing kinematic models, but these methods prove to be either very imprecise or too time-consuming for a practical use.

The invention improves this situation.

### PRESENTATION OF THE INVENTION

The present invention therefore relates, according to a first aspect, to a method for determining parameters for fitting prosthesis on a joint of a patient, the method being characterized in that it comprises the implementation by data processing means of a server of the following steps:
(a) Obtaining a kinematic model of said joint before the prosthesis is fitted, referred to as the preoperative kinematic model;
(b) Constructing a kinematic model of said joint after the prosthesis has been fitted, referred to as the postoperative kinematic model, for current values of the fitting parameters of said prosthesis, by modifying said preoperative kinematic model;
(c) Predicting the postoperative values of a set of functional metrics of said joint depending on said postoperative kinematic model;
(d) Validating or not the current values of the fitting parameters of said prosthesis depending on said predicted postoperative values of the set of functional metrics;
(e) If the current values of the fitting parameters of said prosthesis are not validated, modifying the current values of the fitting parameters of said prosthesis, updating the postoperative kinematic model, and then repeating steps (c) and (d) on the basis of the updated postoperative kinematic model.

According to advantageous and non-limiting features:

Step (a) comprises a sub-step (a1) of obtaining at least one three-dimensional medical image of said joint; and (a2) of constructing said preoperative kinematic model from said three-dimensional medical image of said joint.

Sub-step (a2) comprises identifying the osseous surfaces of said joint in said three-dimensional medical image of said joint.

Said preoperative kinematic model is constructed by finite element biomechanical simulation or by geometric biomechanical simulation from the shapes of the identified osseous surfaces of said joint.

Said preoperative kinematic model is constructed by geometric biomechanical simulation from the shapes of the identified osseous surfaces of said joint, step (b) comprising modifying the shapes of the osseous surfaces of said joint, for the current values of the fitting parameters of said prosthesis, and constructing said postoperative kinematic model by geometric biomechanical simulation from the modified shapes of the osseous surfaces.

Step (a1) comprises acquiring said three-dimensional medical image of said joint by a medical imaging device.

Step (d) compares the predicted postoperative values of the set of functional metrics and the target values of the set of functional metrics.

Step (a) comprises a step (a3) of estimating the preoperative values of said set of functional metrics of said joint based on said preoperative kinematic model, said target values being the estimated preoperative values.

The current values of the fitting parameters of said prosthesis are validated when they minimize a cost function comparing the predicted postoperative values and the target values of the set of functional metrics, the current values of the fitting parameters of said prosthesis being modified in step (e) by a gradient descent type algorithm on said cost function.

Said set of functional metrics defines a ligament balancing of said joint, the functional metrics being in particular variation profiles of the lengths of the ligaments of said joint, called anisometry profiles, or—more simply—anisometries.

Said cost function is F(P₁, . . . P_{N})=ΣᵢΣ_{α}[L_{POST}(P₁, . . . P_{N}, i, α)−L_{PRE}(i, α)]², where L_{PRE}(i, α) and L_{POST}(P₁, . . . P_{N}, i, α) are respectively the preoperative and postoperative lengths of the i-th ligament of said joint for an angle α of flexion of said joint.

The construction of the preoperative or postoperative kinematic model by geometric biomechanical simulation comprises a pre-calculation part aimed at defining an anatomical reference frame from the shapes of the osseous surfaces of said joint, and a part for calculating the kinematics.

The updating of the postoperative kinematic model in step (e) comprises repeating steps (b1) and (b2) for the modified current values of the fitting parameters of said prosthesis.

At least one occurrence of the repeated step (b2) does not comprise the pre-calculation part aimed at defining an anatomical reference frame from the shapes of the osseous surfaces of said joint.

Said geometric biomechanical simulation is solely a function of the identified shapes of the osseous surfaces of said joint.

According to a second aspect, the invention concerns a server for determining parameters for fitting a prosthesis on a joint of a patient, characterized in that it comprises data processing means configured to:
Obtain a kinematic model of said joint before the prosthesis is fitted, referred to as the preoperative kinematic model;
Construct a kinematic model of said joint after the prosthesis has been fitted, referred to as the postoperative kinematic model, for current values of the fitting parameters of said prosthesis, by modifying said preoperative kinematic model;
Predict postoperative values of a set of functional metrics of said joint depending on said postoperative kinematic model;
Validate or not the current values of the fitting parameters of said prosthesis depending on said predicted postoperative values of the set of functional metrics;
If the current values of the fitting parameters of said prosthesis are not validated, modify the current values of the fitting parameters of said prosthesis, update the postoperative kinematic model, and then repeat the steps of predicting the postoperative values of a set of functional metrics of said joint, and validating or not the current values of the fitting parameters of said prosthesis, on the basis of the updated postoperative kinematic model.

According to a third aspect, the invention concerns a system comprising a server according to the second aspect and a medical imaging device, the data processing means being further configured to:
Obtain from said medical imaging device at least one three-dimensional medical image of said joint;
Construct said preoperative kinematic model from said three-dimensional medical image of said joint.

According to a fourth and a fifth aspect, the invention concerns a computer program product comprising code instructions for executing a method according to the first aspect for determining parameters for fitting a prosthesis on a joint of a patient; and a storage medium readable by computer equipment on which is recorded a computer program product comprising code instructions for executing a method according to the first aspect for determining parameters for fitting a prosthesis on a joint of a patient.

### PRESENTATION OF THE FIGURES

Other features and advantages of the present invention will become apparent from the following description of a preferred embodiment. This description will be given with reference to the appended drawings in which:

FIG. 1 is a diagram of a system for implementing the method according to the invention;

FIG. 2 is a flowchart illustrating the steps of an embodiment of the method according to the invention;

FIG. 3a shows a view of the posterior medial condyle of the femur bearing the trace of the contact point of the tibia on the femur during flexion in an example of constructing a preoperative kinematic model by geometric biomechanical simulation;

FIG. 3b shows the discretization of the open contour I defining the trace of the contact point of the tibia on the femur during flexion of FIG. 3a;

FIG. 4 represents an example of a postoperative kinematic model in which the implanted prosthesis and ligaments are shown (the 3 fibers of the medial collateral ligaments are intentionally shown over the bone);

FIG. 5a is a graph representing the estimated preoperative anisometries for an example of joint when said preoperative kinematic model is constructed by biomechanical simulation using finite elements;

FIG. 5b is a graph representing the estimated preoperative anisometries for said example of joint when said preoperative kinematic model is constructed by geometric biomechanical simulation (used for the following figures);

FIG. 5c is a graph representing the predicted postoperative anisometries for said example of joint for the postoperative kinematic model constructed for the initial values of the fitting parameters of said prosthesis;

FIG. 5d is a graph representing the predicted postoperative anisometries for said example of joint for the postoperative kinematic model constructed for the determined optimal values of the fitting parameters of said prosthesis.

### DETAILED DESCRIPTION

### Architecture

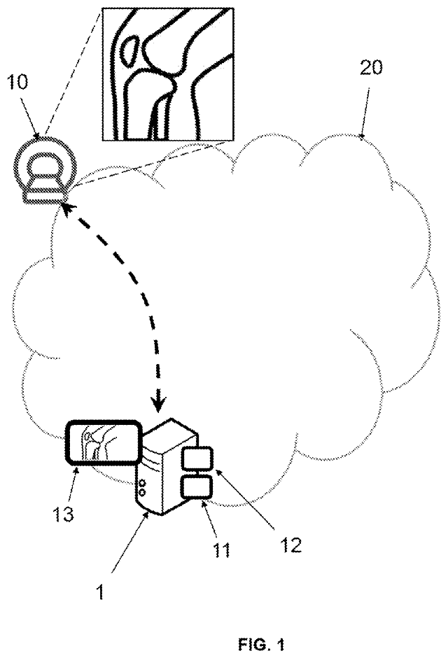

The present invention concerns a method for determining parameters for fitting a prosthesis on a joint of a patient (i.e. fitting parameters of the prosthesis) in a system as shown in FIG. 1. The joint, or articulation is an anatomical element of the patient's body forming a junction zone between multiple bone ends and providing at least one degree of freedom. Said joint 1 is typically the knee (which involves the femur, tibia, and patella), but it can also be the hip, ankle, elbow, wrist, shoulder, etc. In particular, the joint can assume a posture (or pose) from among a plurality of possible postures, typically defined by one or more parameters—called “degrees of freedom”—such as angles, each corresponding to a possible movement of the joint. For example, the knee can thus produce the following movements:
Extension/flexion of the leg on the thigh (approximately 160° range);
Internal/external rotation of the leg on the thigh (approximately 20° range with the knee flexed);
Anteroposterior translation of the tibial plateau (approximately 10 mm range during the extension/flexion movement).

In the remainder of this description, the example of the extension/flexion movement of the knee will be used, but those skilled in the art can apply the invention to other joints and other movements.

The prosthesis in question is typically a total knee prothesis, known as a TKP.

The term «fitting parameter of a prosthesis» broadly refers to any numerical parameter allowing to define the final result after the prosthesis is fitted in the joint during surgery on said joint, in particular a thickness, a position, an angle, an offset, etc. These parameters are «internal», that is to say they relate to the choice and design of the prosthesis (for example, a thickness; a parameter can even be the identifier of a prosthesis model among several possible models), and/or «external», that is to say they relate solely to the fitting conditions, independent of the design (for example, an offset). This is also referred to as «surgical planning».

For example, in the case of a TKP, these fitting parameters may be the following: thicknesses of the distal and posterior resections, axial rotations, frontal rotations in varus or valgus, posterior slopes, and other possible offsets along the three axes.

Naturally, each prosthesis will have its own fitting parameters, and those skilled in the art will be able to apply the present invention to any prosthesis of any joint with any chosen fitting parameters. Moreover, it is entirely possible to manually determine certain fitting parameters, and therefore to apply the present invention only to certain other parameters.

The present method is implemented by a server 1 having data processing means 11 (typically a processor), and generally data storage means 12 (a memory, for example a hard drive) and an interface 13 (for example a screen, a keyboard, an input port, etc.).

Preferably, a medical imaging system 10 is also used to acquire medical images of the joint.

This system 10 can be directly or indirectly (for example via a network 20 such as the internet) connected to the server 1 so that the latter is able to receive the medical images. The interface 13 of the server 1 can also serve as an interface for the system 10 (to control it and obtain the acquired medical images).

Said medical images are typically 3D volumetric images, optionally reconstructed from 2D slices, i.e., tomographic images or «CT scans» (the system 10 is typically an X-ray scanner-CT (computed tomography)). It should be noted that we are not limited to a particular technology and the system 10 could be an MRI, an ultrasound scanner, a PET scanner, etc.

### Method

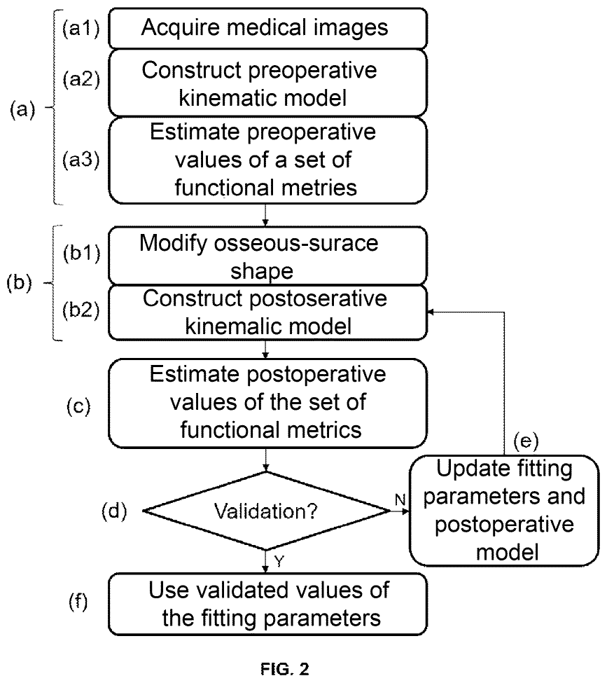

Referring to FIG. 2, the present method is implemented by the data processing means 11 of the server 1, and begins with a step (a) of obtaining a kinematic model of the joint before the prosthesis is fitted, called the preoperative kinematic model.

By «biomechanical model of a joint», or «digital twin», is meant a multidimensional object (in particular two-dimensional or three-dimensional, and preferably three-dimensional) that is articulated, that is to say, movable in the same way as the joint being modeled (and comprising all or part of the degrees of freedom of the joint). The model optionally comprises deformable parts (corresponding in particular to fat, muscles, or ligaments, which can be represented by volumetric meshes (for example, finite element discretizations), and non-deformable parts (corresponding in particular to bones) which can be represented by 3D surface meshes.

Within the scope of the present invention, the kinematic model of said joint can be directly provided to the means 11, but alternatively it is generated.

Step (a) then comprises a sub-step (a1) of obtaining at least one three-dimensional medical image of said joint; and (a2) of constructing said preoperative kinematic model from said three-dimensional medical image of said joint.

Step (a1) may itself comprise the acquisition of said three-dimensional medical image of said joint (and in particular a set of images, preferably representing said joint in a plurality of postures as mentioned above) by the medical imaging device 10.

Step (a2) may comprise the localization of remarkable anatomical points and directions (or «landmarks», see again application FR2300597 on this subject). Typically, in the case of the knee, it is possible to use all or part of the following points, using any morphological analysis of the bone known to those skilled in the art:
The midpoint of the tibial spines, called the tibial center, denoted «TibCenter»;
The center of the two malleoli, called the ankle center, denoted «AnkleCenter»;
The anterior extremity of the anterior tibial tuberosity, denoted «TTA»;
The bottoms of the left and right tibial glenoids, denoted «TibGlen_L and TibGlen_R»;
The hip center, denoted «HipCenter»;
The center of the femoral arch, called the femoral center, noted «FemCenter»;
The internal insertion of the medial collateral ligament on the femur, denoted «FemMCL»;
The insertions of the three fibers (anterior, posterior, and intermediate) of the lateral collateral ligament on the femur, denoted «FemLCL_A, FemLCL_P and FemLCL_I»;
The internal insertion of the medial collateral ligament on the tibia, denoted «TibMCL»;
The insertions of the three fibers (anterior, posterior, and intermediate) of the lateral collateral ligament on the tibia, denoted «TibLCL_A, TibLCL_P and TibLCL_I»;
The posterior femoral condyles on the right and left sides, denoted «LeftPostCond» and «RightPostCond».

The femoral arch is also called the intercondylar notch (femoral notch). It is the bony arch, in axial view, that connects the medial condyle to the lateral condyle. The so-called intercondylar space under the femoral arch contains (among other things) the anterior and posterior cruciate ligaments.

As will be explained, sub-step (a2) mainly comprises the identification of the osseous surfaces of said joint (including the articular surfaces, that is, the parts of the osseous surfaces that come into contact with another bone to form a joint) in said three-dimensional medical image of said joint, it being understood that the bone is generally covered by cartilage, particularly at the joints, and therefore, theoretically, one should speak of a cartilaginous surface, but for convenience, we will not distinguish between bone and cartilage and will generally use the term osseous surface. The importance of having these articular and osseous surfaces is understandable, since it is at their level that the prosthesis will be fitted.

To do this, starting from a three-dimensional medical image, a segmentation method can be implemented to generate a mesh (consisting of facets, particularly triangular ones) representing the external surface of the bones (whose articular surfaces are the mating parts). A mesh normalization phase is advantageously implemented to eliminate topological artifacts (holes or inconsistencies related to the connections between the mesh vertices) or geometric artifacts (inconsistencies in the orientation of the normals, facet inversions, or intersections of the mesh edges).

In contrast to known construction techniques, and in particular the technique defined in application FR2300597 in which the joint kinematics are actually observed through the use of a plurality of medical images associated with a plurality of postures, the kinematics here will be simulated.

Said preoperative kinematic model is thus constructed by geometric biomechanical simulation from the shapes of the osseous surfaces of said joint that have been identified.

The geometric simulation makes it possible to calculate the relative movement of two bones by considering only the shape of the articular surfaces in contact. To rephrase, it is a biomechanical simulation that makes it possible to construct a kinematic model (therefore related to movement) of the joint by direct geometric processing of the shapes of the osseous surfaces of said joint, and preferably only depending on said shapes of the osseous surfaces of said joint. It is thus understood that the geometric simulation is potentially limited to the surfaces and ignores the volumes and various tissues that fill them.

It is thus understood that the geometric simulation only allows the calculation, from the 3D surface models of the involved organs, of the relative positions of the anatomical structures during a joint movement, and in this respect, it differs from classical biomechanical simulation, which allows the simulation (in addition to kinematic variables) of kinetic variables (i.e., related to the forces involved during the movement), such as finite element biomechanical simulation, which is a reference method.

The latter allows the simulation of an active movement of the joint as it would be produced by a conscious contraction of the muscles of the limb, but it is complex because it uses a numerical simulation environment to represent the studied anatomical structures (bones, ligaments, and muscles) within the patient's biomechanical model and to reproduce the boundary conditions that will generate the movement.

It is thus possible to simulate the interactions between non-deformable structures (or «rigid bodies») subjected to forces produced by muscle contractions, and constrained in their movement by the geometry of the patient's articular surfaces and by ligament tensions. Before starting the simulation, the fitting of the ligaments and tendons on the subject under study is carried out by pointing to anatomical points (the «landmarks» mentioned earlier). A Hill model (see the document «M. Millard, T. Uchida, A. Seth and S. L. Delp, “Flexing computational muscle: modeling and simulation of musculotendon dynamics”, Journal of biomechanical engineering, 135 (2), 2013») is then used, for example, to describe the muscle fibers that produce the movement (quadriceps and its hamstring antagonists for leg flexion), whose path is based on the landmarks.

The result of the simulation is advantageously a time series of 4×4 rigid transformation matrices, expressed in homogeneous coordinates, giving at each time step, in the case of a flexion/extension movement, the position of the tibia relative to the femur during the movement. The flexion is simulated, for example, for femur-tibia angles varying from 0° to 85° (measured in the sagittal plane). In addition to these so-called «fitting» matrices, finite element analysis also produces fields of numerical values (or tensor fields) allowing quantification of the internal stress state of the biological tissues or modeled mechanical structures (prosthesis).

The geometric simulation is a clever alternative to classic biomechanical simulation because it dispenses with the rheological parameters and complex numerical methods that the latter requires.

Indeed, the geometric approach considers only the shapes of the articular surfaces and formulates hypotheses about the contact between the solids at their point of tangency during the movement. The resulting calculations are therefore several orders of magnitude faster than those required by a biomechanical modeling approach (less than one minute instead of several hours), which makes the geometric approach particularly suitable for interactive exploration of planning parameters. Furthermore, this efficiency makes the method compatible with the constraints of implementation in the operating room, and therefore usable during a surgical procedure.

The algorithm for constructing the kinematic model by geometric biomechanical simulation is preferably divided into two parts: a pre-calculation part aimed at defining an anatomical reference frame from the shapes of the identified osseous surfaces of said joint (and advantageously comprising the definition of an anatomical reference frame for each of the two bones involved in the joint, and the extraction on said surfaces of an open contour defining the trace of a point of contact between said two bones during the flexion of the joint), and a kinematic calculation part (definition of a positioning matrix of one of said bones relative to the other), which we will now describe for the example of the kinematics of the tibia relative to the femur, and which those skilled in the art will know how to adapt to any other joint.

As we will see, this mode of operation is even more efficient, because in case of an update of the kinematic model (for example—in the case of a simulation of a post-operative result—by adjusting the parameters of prosthesis fitting), the pre-calculation only needs to be partially repeated, or not at all (i.e., only the kinematic calculation part needs to be rerun). The operation goes from less than one minute to less than one second.

Thus, not only is geometric biomechanical simulation already tens of times faster than classic biomechanical simulation, but since the pre-calculation phase is the longest, the «simplified» geometric simulation without all or part of the pre-calculation becomes thousands of times faster, and therefore very easily usable in real time, which opens up prospects for combinatorial exploration of parameters and thus the possibility of automatically searching for the optimal therapeutic strategy for a given patient.

### Pre-Calculation

1) Construction of the anatomical reference frame on the tibia, denoted «TibRef», using the anatomical points identified on the bone, with:

$$TibRef = ( TibCenter , \vec{{X}_{T}} , \vec{{Y}_{T}} , \vec{{Z}_{T}} )$$

$$\vec{{X}_{T}} = \vec{AnkleCenter - TibCenter} / ‖ \vec{AnkleCenter - TibCenter} ‖$$

$$\vec{{Z}_{T}} = {\prod }_{{X}_{T} = 0} ( \vec{TTA - TibCenter} ) / ‖ {\prod }_{{X}_{T} = 0} ( \vec{TTA - TibCenter} ) ‖$$

$$\vec{{Y}_{T}} = \vec{{Z}_{T}} \times \vec{{X}_{T}}$$

The operator Π projects the vector onto a plane whose normal is X_{T}. At the end of this calculation, TibRef is centered on the midpoint of the tibial spines, X_{T} points towards the ankle center and defines the «proximal to distal» direction, Y_{T} points towards the patient's left and defines the «right to left» direction, and Z_{T} points towards the patient's front and defines the «posterior to anterior» direction. For all the reference frames described here, the naming of the axes and the conventions for constructing the reference frame may vary, as long as it is possible to unambiguously and reproducibly attach a 3D reference frame to the modeled bone.
2) Construction of the anatomical reference frame on the femur, denoted «FemRef», using the anatomical points identified on the bone, similarly to the tibia:

$$FemRef = ( FemCenter , \vec{{X}_{F}} , \vec{{Y}_{F}} , \vec{{Z}_{F}} )$$

$$\vec{{X}_{F}} = \vec{HipCenter - FemCenter} / ‖ \vec{HipCenter - FemCenter} ‖$$

$$\vec{{Z}_{F}} = \vec{{X}_{F}} \times \vec{RightPostCond - LeftPosCond} / ‖ \vec{{X}_{F}} \times \vec{RightPostCond - LeftPosCond} ‖$$

$$\vec{{Y}_{F}} = \vec{{Z}_{F}} \times \vec{{X}_{F}}$$

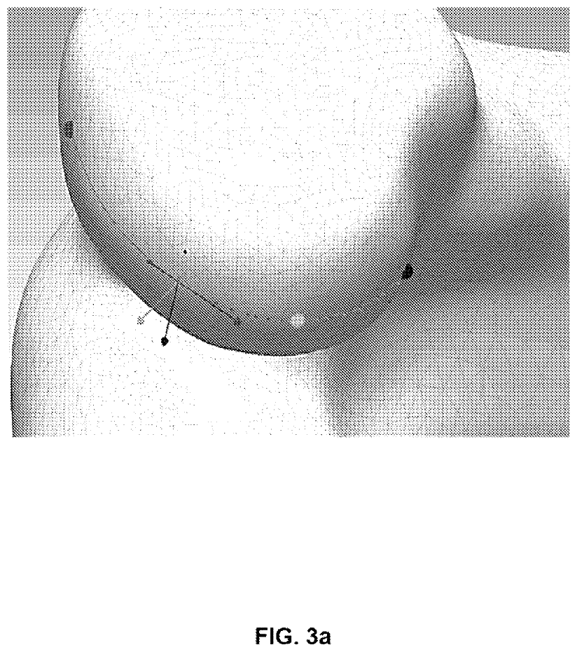

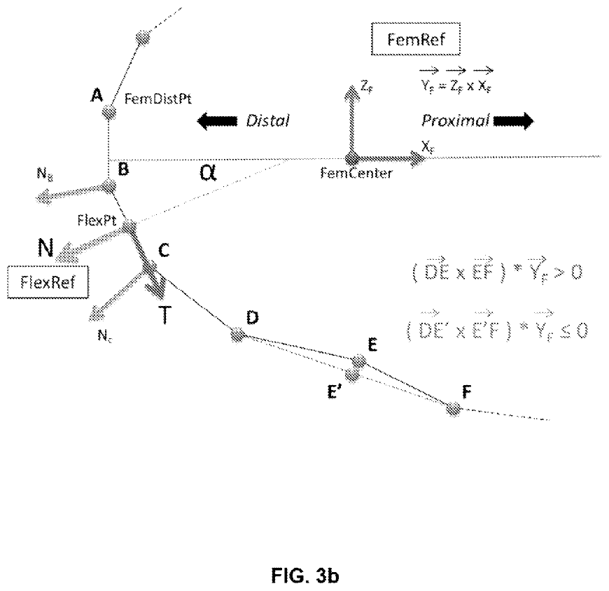

At the end of this calculation, FemRef is centered on the center of the femoral arch, X_{F} points towards the hip center and defines the «distal to proximal» direction, Y_{F} points towards the patient's right and defines the «left to right» direction, and Z_{F} points towards the patient's front and defines the «posterior to anterior» direction.
3) Calculation of the most distal point on the medial condyle of the femur, denoted FemDistPt, which is the medial contact point between the tibia and the femur, with the knee in extension (i.e., for a flexion angle α equal to zero—the flexion angle α expressing the degree of flexion of the tibia relative to the femur, as it would be measured in the patient's sagittal radiological plane). This calculation is performed in FemRef by searching for the point with the minimum coordinate X and whose coordinate Y is negative (for a right knee) or positive (for a left knee).
4) Extraction of the 2D closed contour corresponding to the intersection between the triangular mesh of the femur (see FIG. 3a) and the plane passing through FemDistPt and whose normal is Y_{F}. This algorithm is based on an analysis of the shapes of the identified osseous surfaces of the joint, in particular here the set of facets of the femoral mesh.
5) Extraction of the open sub-contour Γ defining the trace of the tibial contact point on the femur during the flexion. This sub-contour is defined as the part of the closed contour extracted in step 4, which: 1) starts at the distal point FemDistPt, 2) reaches the point with the minimum coordinate along Z_{F}, and 3) ends when this contour rises above the Z_{F}=0 plane.
6) Rectification of Γ to guarantee its convexity. The calculation of the knee flexion angle is based on the assumption that Γ is convex in order to guarantee the uniqueness of the contact point for a given flexion angle. The convexity is corrected by projecting all vertices S of Γ that generate a concavity onto the local convex envelope defined by the segment connecting the preceding vertex S to the vertex following it. This operation is performed iteratively until the contour stabilizes. FIG. 3b illustrates this calculation.
7) Each vertex of the contour Γ is then equipped with an outward normal calculated by finite differences on the discretized contour (see, for example, points B and C with their normals NB and NC in FIG. 3b). The open contour Γ thus produced carries, during the flexion-extension movement, a sliding Frenet frame denoted «FlexRef», which allows us to position the tibia relative to the femur at each flexion angle, i.e., at each posture.
   Calculation of the Kinematics (Relative Fitting of the Tibia with Respect to the Femur for a Given Flexion Angle α)

With reference to FIG. 3b, the part of the algorithm described here allows us to calculate, for any input flexion angle α, the position of the sliding Frenet frame FlexRef which corresponds, on the contour Γ, to the point of contact between the tibia and the femur.
1 The interval [B, C] containing FlexPt, the origin of the coordinate system FlexRef, is defined as the one where the angles between the «proximal to distal» axis and the normals at B and C bracket the value of α. The position of FlexPt can be calculated by linear interpolation between these three values.
2) The vector N («normal») of FlexRef is the linear interpolation between the two normals at the vertices of Γ that enclose it. The vector T («tangential») of FlexRef is orthogonal to N and contained in the sagittal plane, which is the plane also containing Γ. The vector S («sagittal») of FlexRef (not visible in FIG. 3b because it is in the vertical plane) is calculated by vector product.
3) The kinematic hypothesis is then as follows: for any angle α, the bottom of the medial tibial glenoid is in tangential contact at FlexPt with the contour Γ.
4) The tibia is then equipped with its own coordinate system FlexRef, centered on the bottom of the medial tibial glenoid, and whose axes are oriented in a manner consistent with those of its femoral counterpart (by virtue of the previous kinematic hypothesis).
5) Finally, the matrix that positions the tibia relative to the femur in flexion of α degrees is calculated as the following matrix product:
M_{Tib→Fem}=P_{FemRef→W}×P_{FlexRef→FemRef}×P_{TibRef→FlexRef}×P_{w→TibRef}, where M_{Tib→Fem} is the displacement matrix that positions the tibia relative to the femur in the world coordinate system denoted «W»; P_{FemRef→W} is a transformation matrix that converts the coordinates from the anatomical coordinate system FemRef to the world coordinate system W; P_{FlexRef→FemRef} is the only one of the four transformation matrices that depends on a and it allows conversion from the sliding coordinate system FlexRef to the anatomical coordinate system FemRef; P_{TibRef→FlexRef} is the fixed transformation matrix that transforms from the anatomical reference frame TibRef to the fixed tibial reference frame FlexRef which, during movement, is superimposed with its movable counterpart on the femur; finally, P_{w→TibRef} is the transformation matrix that converts the patient's anatomical coordinates, expressed in the world reference frame W, to the anatomical reference frame TibRef. The four transformation matrices can be easily calculated from the points and vectors defined above.
6) The fitting matrix M_{Tib→Fem} thus obtained for each flexion angle α can then be used to position the tibial insertions during movement and calculate the values of any set of functional metrics such as variations in collateral ligament lengths (anisometries), or the stresses within these ligaments (calculated by means of a finite element model coupled to the geometric model, for example).

Indeed, step (a) preferably comprises in a sub-step (a3) the estimation of the preoperative values (i.e., in the current state, before prosthesis fitting) of a set of functional metrics of said joint in accordance with said preoperative kinematic model.

The term «set of functional metrics», means a group of one or more biomechanical metrics (the set may contain only one functional metric, although it is generally a plurality) functionally describing an aspect of the joint.

Some functional metrics are called «morphological» in that they allow characterizing the shape of a bone before and after prosthesis fitting in order to quantify the geometric difference between the native (preoperative) and prosthetic (postoperative) articular surfaces. The morphological metrics can thus consider differences in distance between the two surfaces or differences in volume by comparing the volume of bone removed with the volume of the prosthesis that replaced it. It is understood that these are indeed functional metrics because the morphology of the joints constrains their function.

According to a preferred embodiment, particularly in the case of the knee, said set of functional metrics defines a ligament balance of said joint, the set of functional metrics being in particular the set of variation profiles of the lengths of the ligaments of said joint, called anisotropies. The ligament balance represents «the balance» in the bilateral tension of the ligaments, and should be disturbed as little as possible by the fitting of the prosthesis.

According to a preferred embodiment, said set of functional metrics defines joint trajectories such as patellar tracking, and said set then consists of the values of the 6 degrees of freedom (3 Euler rotations and 3 translations) at every moment of the movement. Another way to define these successive fittings is to choose 3 (non-collinear) points on the surface of the (triangular) bone model and compare at each instant the position of these 3 points on the «target» bone with the position of these same 3 points on the bone whose trajectory is being optimized. A third way consists of not restricting oneself to 3 points but instead considering all the points of the mesh as reference points. The trajectory optimization will then consist of finding the fitting of the prosthesis that best approximates, at each instant, the positions of all the points of the target mesh at the considered instant.

Other sets of functional metrics may be selected, particularly depending on the risks and objectives of the prosthesis fitting. Several sets of functional metrics may even be selected and combined, for example, by measuring at each instant the elongation rate of ligament fibers, or by measuring at each instant the internal stresses within tissues whose exposure to excessive internal forces is to be limited. Generally speaking, any quantity reflecting a degree of adequacy or inadequacy between the current state of the model and a functional objective can be used. Measures of inadequacy should then be minimized, while measures of adequacy should be maximized.

In all cases, the preoperative values of functional metrics can be easily read from the preoperative model, and are preferably used as «target» values, i.e., values to be approximated as closely as possible in the postoperative state (which would indicate an absence of excessive disturbance of the surrounding soft tissues of the knee between before and after surgery). Alternatively, one may wish to improve an existing situation (if, for example, the joint presents a pathology) through the fitting of the prosthesis, and therefore one has values calculated from the preoperative values, but different from the latter, or even have predetermined target values and avoid this step of estimating preoperative values. Alternatively, they can also be measured on the patient using ancillary devices or 3D tracking equipment.

### Postoperative Prediction

The idea behind the present invention is, instead of attempting to directly determine optimal values for the fitting parameters of the prosthesis, which proves impossible in practice, to proceed by iterations and to converge towards these optimal values (within a clinically acceptable set of values).

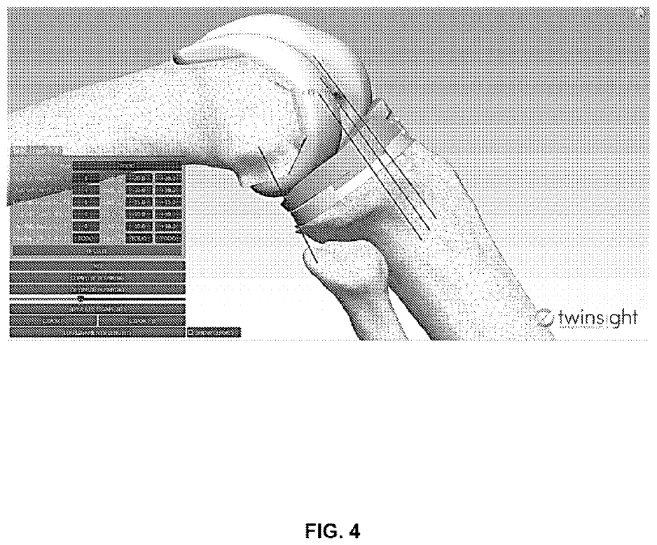

As such, in step (b), the means 11 construct a kinematic model of the joint after prosthesis fitting, called the postoperative kinematic model, for current values of the fitting parameters of the prosthesis, by modifying the preoperative kinematic model. To rephrase, the prosthesis fitting will be simulated using the preoperative kinematic model. To do this, one can simply implement a substitution of a portion of the osseous surface of the preoperative model with a volume representing the prosthesis, determined by the aforementioned fitting parameters, as shown, for example, in FIG. 4. Said «current» values are intended to initialize the method and are typically either default values or candidate values proposed by the surgeon, potentially already close to the optimal values, which shortens the method, but they could also be arbitrary values.

Step (b) can thus involve geometric modeling, the principles of which have been described above and which is translated immediately to the postoperative state by simply replacing the patient's preoperative articular surfaces with the prosthetic articular surfaces. To rephrase, in a sub-step (b1), «modified» osseous surfaces of the joint are determined for the current values of the fitting parameters of the prosthesis, corresponding to the osseous surfaces fitted with the prosthesis. The postoperative kinematic model is then constructed in a sub-step (b2) by a new geometric biomechanical simulation based on the shapes of the modified osseous surfaces. It is understood that there is construction of a complete new kinematic model (potentially reusing all or part of the pre-calculations of the relative displacements between the modeled bone, tissue, and/or prosthetic entities), but since the geometric biomechanical simulation is very fast, there is no problem in repeating it completely based on the modified osseous surfaces.

Then, in step (c), post-operative values are predicted for the set of functional metrics of the joint based on the post-operative kinematic model. This step is typically implemented in the same way as the possible step (a3) of estimating the pre-operative values of the set of functional metrics of the joint based on the pre-operative kinematic model, the difference being that we are working on the modified model that includes the prosthesis.

This step is referred to as a prediction since the post-operative kinematic model remains theoretical, even if so-called validation and verification techniques allow the models used to be calibrated and certified in order to guarantee high reliability in the representation of the modeled structures.

Then, in step (d), means 11 validate or not the current values of the fitting parameters of the prosthesis based on the predicted post-operative values of the set of functional metrics. The idea is that the current values of the fitting parameters are acceptable if the post-operative values of the metrics are themselves acceptable.

Typically, step (d) compares the predicted post-operative values of the set of functional metrics and the target values, in particular the pre-operative values or predefined values.

Any mathematically relevant criterion can be used for this measurement of the difference between the current values and the target values, for example, an error rate, a maximum quadratic distance, etc. A preferred embodiment will be described further below.

If the current values of the fitting parameters of the prosthesis are validated (for example, because the predicted postoperative values of the set of functional metrics are sufficiently close to the target values), then the method can stop. The method may, for example, comprise a step (f) of using the validated values of the fitting parameters in a medical application (for example, passive surgical navigation or a robotic surgical procedure). Step (f) may comprise, for example, displaying the validated current values on the interface 13 for the surgeon, who then knows that they can apply them, and/or sending the parameters to means for generating the prosthesis (for example, a 3D printer, a machining equipment, etc.) based on the parameters.

Step (f) may also comprise the actual fitting of the prosthesis on the patient during a surgical operation.

Otherwise, if the current values of the fitting parameters of the prosthesis are not validated, the method comprises a step (e) of modifying the current values of the fitting parameters of the prosthesis (if necessary, with the possibility of locking certain parameters, either because their current value is the desired one, or because it is not desired for the algorithm to propose values outside a predetermined range of acceptable values), updating the postoperative kinematic model, and then repeating steps (c) and (d) on the basis of the updated postoperative kinematic model.

In other words, step (b) is repeated not based on the preoperative model, but on the «unvalidated» postoperative model, using new parameters, and steps (c) and (d) are repeated until validated values for the fitting parameters are found (and step (f) is implemented). It should be noted that, in the case of a new implementation of step (b2), no new pre-calculation is needed as long as only external fitting parameters are modified, because the relative movement of the prosthetic articular surfaces is intrinsic to the prosthesis and therefore does not depend on its fitting position within the joint space. The implementation of numerous iterations is therefore extremely fast. And even if a pre-calculation is still necessary in some iterations (for example, when testing a different prosthesis model), this only concerns a fraction of the iterations, and the geometric simulation itself is already extremely fast, thus compatible with the time constraints of surgical procedures in the operating room.

To achieve «optimal» fitting parameters, any known optimization algorithm can be implemented: gradient descent, stochastic methods, genetic algorithms, etc. One could even, naively, randomly test new values for the fitting parameters.

In a particularly preferred embodiment, the current values of the fitting parameters of the prosthesis are validated when they minimize a cost function comparing the predicted postoperative values and the target values of the set of functional metrics.

This allows the current values of the fitting parameters of said prosthesis to be modified in step (e) by a gradient descent algorithm on the cost function, which guarantees a convergent behavior.

For example, in the case where the set of functional metrics defines a ligament balancing, the cost function is advantageously the sum over all the ligaments of the joint of the squared differences between the predicted postoperative values and the target values of the anisotropies, and in particular, in the case of the knee, according to the formula: F(P₁ . . . P_{N})=Σ_{L}Σ_{α}[Lₚₒₛₜ(P₁ . . . P_{N}, L, α)−L_{Pré}(L, α)]², where P₁, . . . , P_{N} denote the fitting parameters, L=1, 2, 3 or 4 denotes the four collateral ligaments, α is an index that designates the posture (or directly a flexion angle—typically from 0° to 70°), L_{Pré}(L, α) is the preoperative length of the ligament L at the posture α and Lₚₒₛₜ(P₁ . . . P_{N}, L, α) is the corresponding postoperative length for the fitting parameters P1, . . . , PN.

It should be noted that in the implementation example given here, to arrive at four collateral ligaments of the knee, in practice the lateral collateral ligament (LCL) is represented by a single fiber, while the medial collateral ligament (MCL) is divided into three fibers, anterior (aMCL), intermediate (iMCL) and posterior (pMCL), which exhibit distinct anisometric tendencies.

Alternatively, in the case where said set of functional metrics defines patellar kinematics, said cost function may be the sum of the squares of the differences between the predicted postoperative degrees of freedom (spatial translations and rotations) and the target values of these degrees of freedom. Another possible formulation could be based on the calculation of a Hausdorff distance (symmetric or not) between a 3D surface model of the patella in its predicted postoperative fitting and the same model in the target postoperative fitting.

### Results

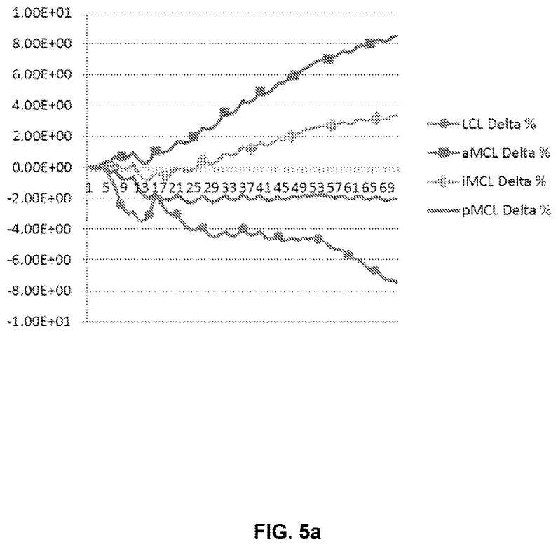

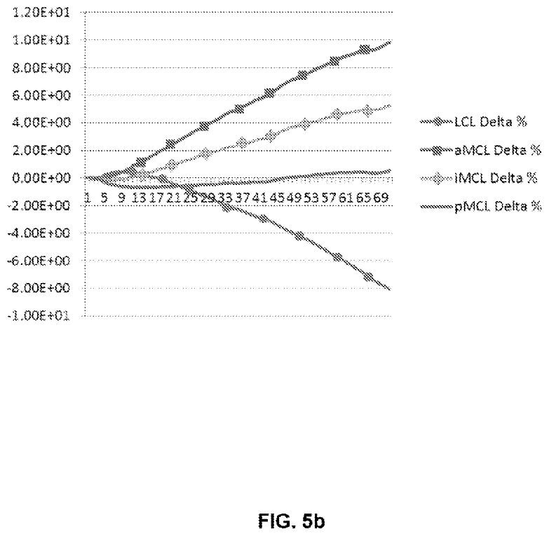

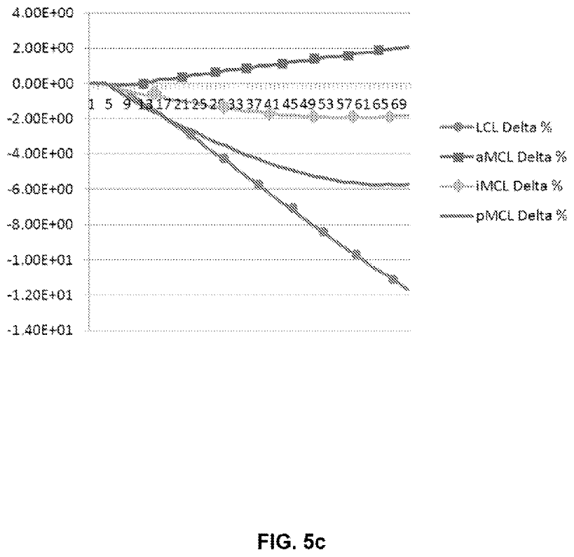

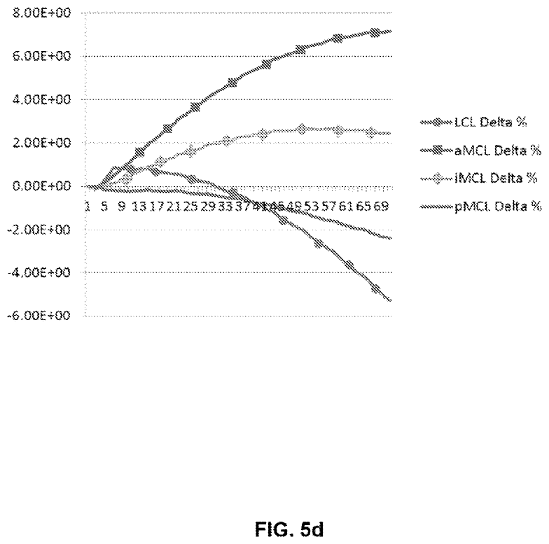

FIGS. 5a to 5d demonstrate the effectiveness of the present method on an example of TKP fitting. The qualitative comparison consists of validating the lengthening or shortening tendencies of the four aforementioned collateral ligament fibers (LCL, aMCL, iMCL and pMCL) in order to validate that the model predicts a consistent trend.

FIG. 5a and FIG. 5b first represent the estimated preoperative anisotropies for an example joint when said preoperative kinematic model is respectively constructed by finite element biomechanical simulation (as a reference method against which the geometric simulation is evaluated) and by geometric biomechanical simulation, expressed depending on the angle α in percentage of elongation relative to the reference length of the ligament (when the knee is in extension, i.e., α=0°).

The results of the two models are not identical but show exactly the same trends of lengthening or shortening, which is a critical qualitative result in therapeutic decision-making during a surgical procedure, even though the geometric biomechanical simulation is tens or even thousands of times faster (in the absence of new pre-calculation).

Based on the preoperative kinematic model constructed by geometric biomechanical simulation (that of FIG. 5b), the first iteration of the postoperative kinematic model construction was initially implemented, i.e., step (b), based on initial values of the prosthesis fitting parameters, provided by an expert operator, as current values, and the prediction of the corresponding postoperative values of the anisometries (first occurrence of step (c)). The result, visible in FIG. 5c, shows initially non-physiological anisometric trends (large deviations from FIG. 5b), so these current values are not validated (step (d)).

In accordance with step (e), the current values of the prosthesis fitting parameters are optimized, the postoperative kinematic model is updated, and steps (c) and (d) are repeated based on the updated postoperative kinematic model.

After a few iterations, a clear evolution of the anisometric trends is observed, which, at the end of the optimization, are very close to the natural anisometries of the knee, see FIG. 5d (compare with FIG. 5b). The residual error is 2.51 mm on average over the four insertion points and over the entire range of motion. This residual is mainly due to the mechanical design of the prosthesis and is therefore difficult to reduce.

In terms of performance, the pre-calculations necessary for the construction of the pre- and postoperative kinematic models take approximately 2 seconds. The kinematics of the preoperative or postoperative knee can then be simulated in 1.5 milliseconds. A gradient descent optimization of 2000 iterations results in optimal values for the fitting parameters where the average deviation in the position of the tibial insertions over the entire flexion-extension movement is 2.51 mm (as explained above). With a geometric biomechanical simulation, this calculation requires the evaluation of anisotropies for approximately 20,000 sets of current values of the fitting parameters (in order to calculate the gradient by finite differences at each step) but takes only 30 seconds, whereas a conventional biomechanical simulation would require many minutes or even hours, thus making it unusable in the operating room.

### Server

According to a second aspect, the invention concerns the server 1 for implementing the method according to the first aspect.

Thus, this server 1 comprises, as explained, at least data processing means 11 and a memory 12. The server may also comprise an interface 13 such as a screen. It is typically a server for determining the fitting parameters of a prosthesis on a joint of a patient.

The data processing means 11 are configured to implement steps consisting of:
Obtaining a kinematic model of said joint before the prosthesis is fitted, referred to as the preoperative kinematic model (obtaining at least a three-dimensional medical image of said joint; then identifying osseous surfaces of said joint in said three-dimensional medical image of said joint, and constructing, by geometric biomechanical simulation from the shapes of the identified osseous surfaces of said joint, the preoperative kinematic model);
Constructing a kinematic model of said joint after the prosthesis has been fitted, referred to as the postoperative kinematic model, for current values of the prosthesis fitting parameters, by modifying the preoperative kinematic model (modify the shapes of the osseous surfaces of said joint for the current values of the prosthesis fitting parameters; and construct, through geometric biomechanical simulation based on the modified shapes of the osseous surfaces, the postoperative kinematic model for the current values of the fitting parameters of said prosthesis);
Predicting postoperative values of a set of functional metrics of said joint based on said postoperative kinematic model;
Validating or not the current values of the fitting parameters of said prosthesis depending on said predicted postoperative values of the set of functional metrics;
If the current values of the fitting parameters of said prosthesis are not validated, modifying the current values of the fitting parameters of said prosthesis, updating the postoperative kinematic model, and then repeating the steps of updating the postoperative kinematic model, and then repeating the steps of predicting the postoperative values of a set of functional metrics of said joint, and validating or not the current values of the fitting parameters of said prosthesis, on basis of the updated postoperative kinematic model.

According to a third aspect, the invention provides a system comprising said server 1, as well as a connected medical imaging device 10 (via the network 20).

The data processing means 11 are then further configured to:
Obtain from said medical imaging device 10 at least one three-dimensional medical image of said joint;
Construct said preoperative kinematic model from said three-dimensional medical image of said joint.

The data processing means 11 may also be configured to:
Estimate preoperative values of said set of functional metrics of said joint depending on said preoperative kinematic model; or
Use the validated fitting parameter values, for example to manufacture the prosthesis by 3D printing.

### Computer Program Product

According to a fourth and a fifth aspect, the invention concerns a computer program product comprising code instructions for executing (on the data processing means 11 of the server 1) a method according to the first aspect for determining parameters for fitting a prosthesis on a joint of a patient, as well as storage means readable by computer equipment (for example, the data storage means 12 of the server 1) on which this computer program product is stored.

1. A method for determining parameters for fitting prosthesis on a joint of a patient, the method being wherein it comprises the implementation by data processing means of a server of the following steps:
(a1) obtaining at least one three-dimensional medical image of the joint;
(a2) identifying the osseous surfaces of the joint in the three-dimensional medical image of the joint, and constructing, by geometric biomechanical simulation from the shapes of the identified osseous surfaces of the joint, a kinematic model of the joint before the prosthesis fitting, referred to as preoperative kinematic model;
(b1) modifying the shapes of the osseous surfaces of said joint, for current values of the fitting parameters of the prosthesis;
(b2) constructing, by geometric biomechanical simulation from the modified shapes of the osseous surfaces, a kinematic model of the joint after prosthesis fitting, the postoperative kinematic model, for the current values of the fitting parameters of said prosthesis;
(c) predicting the postoperative values of a set of functional metrics of the joint depending on said postoperative kinematic model;
(d) validating or not the current values of the fitting parameters of the prosthesis depending on the predicted postoperative values of the set of functional metrics;
(e) if the current values of the fitting parameters of the prosthesis are not validated, modifying the current values of the fitting parameters of the prosthesis, updating the postoperative kinematic model, and then repeating steps (c) and (d) on the basis of the updated postoperative kinematic model.

2. The method according to claim 1, wherein step (a1) comprises acquiring the three-dimensional medical image of the joint using a medical imaging device.

3. The method according to claim 1, wherein step (d) compares the predicted postoperative values of the set of functional metrics and the target values of the set of functional metrics.

4. The method according to claim 3, wherein step (a) comprises a sub-step (a3) of estimating the preoperative values of the set of functional metrics of the joint depending on the preoperative kinematic model, the target values being the estimated preoperative values.

5. The method according to claim 3, wherein the current values of the fitting parameters of the prosthesis are validated when they minimize a cost function comparing the predicted postoperative values and the target values of the set of functional metrics, the current values of the fitting parameters of the prosthesis being modified in step (e) by a gradient descent type algorithm on the cost function.

6. The method according to claim 1, wherein the construction of the preoperative or postoperative kinematic model by geometric biomechanical simulation comprises a pre-calculation part aimed at defining an anatomical reference frame from the shapes of the osseous surfaces of the joint, and a part for calculating the kinematics.

7. The method according to claim 1, wherein the updating of the postoperative kinematic model in step (e) comprises repeating steps (b1) and (b2) for the modified current values of the fitting parameters of the prosthesis.

8. The method according to claim 6 in combination, in which at least one instance of the repeated step (b2) does not comprise the pre-calculation part aimed at defining an anatomical reference frame from the shapes of the osseous surfaces of the joint.

9. The method according to claim 1, wherein the geometric biomechanical simulation is solely based on the identified shapes of the osseous surfaces of the joint.

10. A server for determining parameters for fitting a prosthesis on a joint of a patient, wherein it comprises data processing means configured to:
obtain at least one three-dimensional medical image of the joint;
identify the osseous surfaces of the joint in the three-dimensional medical image of the joint, and construct, by geometric biomechanical simulation based on the identified osseous surface shapes of the joint, a kinematic model of the joint before prosthesis fitting, referred to as the preoperative kinematic model;
modify the osseous surface shapes of the joint for current values of the prosthesis fitting parameters;
construct, by geometric biomechanical simulation based on the modified osseous surface shapes, a kinematic model of the joint after prosthesis fitting, referred to as the postoperative kinematic model for current values of the prosthesis fitting parameters;
predict postoperative values of a set of functional metrics of the joint based on the postoperative kinematic model;
validate or not the current values of the fitting parameters of the prosthesis depending on the predicted postoperative values of the set of functional metrics;
if the current values of the fitting parameters of the prosthesis are not validated, modify the current values of the fitting parameters of the prosthesis, update the postoperative kinematic model, and then repeat the steps of predicting the postoperative values of a set of functional metrics of the joint, and validating or not the current values of the fitting parameters of the prosthesis on the basis of the updated postoperative kinematic model.

11. A system comprising a server according to claim 10 and a medical imaging device, the data processing means being further configured to:
obtain from the medical imaging device at least one three-dimensional medical image of the joint;
construct the preoperative kinematic model from the three-dimensional medical image of the joint.

12. A computer program product comprising code instructions for executing a method according to claim 1 for determining parameters for fitting a prosthesis on a joint of a patient, when the program is executed on a computer.

13. A storage means readable by a computer equipment on which is stored a computer program product comprising code instructions for executing a method according to claim 1 for determining parameters for fitting a prosthesis on a joint of a patient.
